(19) United States
(12) Patent Application Publication
(10) Pub. No.: US 2024/0306934 A1
(43) Pub. Date: Sep. 19, 2024

(54) RESPIRATION MONITORING

(71) Applicant: Welch Allyn, Inc., Skaneateles Falls, NY (US)

(72) Inventors: WonKyung McSweeney, Manlius, NY (US); John A. Lane, Venice, FL (US); David E. Quinn, Moravia, NY (US); Tyson B. Whitaker, Arden, NC (US); Gene J. Wolfe, Pittsford, NY (US)

(21) Appl. No.: 18/588,771

(22) Filed: Feb. 27, 2024

Related U.S. Application Data
(60) Provisional application No. 63/489,901, filed on Mar. 13, 2023.

(51) Int. Cl. A61B 5/08; A61B 5/00; A61B 5/091

(52) U.S. Cl. CPC A61B 5/0816; A61B 5/0024; A61B 5/091; A61B 5/6802

(57) ABSTRACT

Systems for monitoring respiration are described. One system receives spatiotemporal data from a sensor array worn by a patient. The spatiotemporal data includes spatial and temporal components. The system feeds the spatiotemporal data into a machine learning model. The system characterizes the respiration based on an output of the machine learning model. Another system identifies one or more edges on a wearable article, and tracks a frequency of motion of the one or more edges on the wearable device to determine the respiration rate.

### BACKGROUND

In some instances, a wearable device can be used to monitor respiration of a subject, such as a patient admitted to a healthcare facility. When monitoring respiration with a wearable device, the performance of the device depends on the position and movement of the subject. This is typically due to limited information collected from a single spot on the subject's body.

Video-based monitoring is one way to monitor vital signs, such as respiration rate, without having to physically contact the subject. For example, close-up video feeds of a subject's face can provide enough information to monitor various vital signs. However, in clinical healthcare settings, many environmental factors such as distance, angle, and lighting can make video-based monitoring of patient vital signs challenging.

### SUMMARY

In general terms, the present disclosure relates to monitoring respiration. In one possible configuration, data having both spatial and temporal components is used to characterize respiration. In another possible configuration, a wearable article and/or a structured light provide a pattern that assists video-based respiration monitoring. Various aspects are described in this disclosure, which include, but are not limited to, the following aspects.

One aspect relates to a system for monitoring respiration, the system comprising: at least one processing device; and at least one computer readable data storage device storing software instructions that, when executed by the at least one processing device, cause the at least one processing device to: receive spatiotemporal data from a sensor array worn by a patient, the spatiotemporal data including spatial and temporal components; feed the spatiotemporal data into a machine learning model; and characterize the respiration based on an output of the machine learning model.

Another aspect relates to a system for monitoring respiration, the system comprising: at least one processing device; and at least one computer readable data storage device storing software instructions that, when executed by the at least one processing device, cause the at least one processing device to: identify one or more edges on a wearable article; and track a frequency of motion of the one or more edges on the wearable device to determine a respiration rate.

Another aspect relates to method of monitoring respiration, the method comprising: searching for edges in a pattern on an article worn over a chest area; evaluating information content of the edges; selecting an edge in the pattern for identifying a local area of the pattern; and monitoring the respiration by tracking a motion frequency of the local area of the pattern.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 illustrates an example of a monitoring system that monitors one or more aspects of a patient including at least the patient's respiration.

FIG. 2 schematically illustrates another example of the monitoring system of FIG. 1.

FIG. 3 shows an example embodiment of a wearable article that can be used by the monitoring system of FIG. 1 to monitor the respiration of the patient.

FIG. 4 graphically illustrates an example of a chart illustrating spatiotemporal data detected by a sensor array of the wearable article of FIG. 3.

FIG. 5 schematically illustrates an example of the monitoring system of FIG. 1 including a respiration monitoring algorithm for monitoring the respiration of the patient.

FIG. 6 graphically illustrates an example of a chart having a bounding box generated as an output from the respiration monitoring algorithm shown in FIG. 5.

FIG. 7 schematically illustrates an example of a method of monitoring respiration performed using the example embodiment of the wearable article shown in FIG. 3.

FIG. 8A shows another example embodiment of the wearable article that can be used by the monitoring system of FIG. 1 to monitor the respiration of the patient, the wearable article is shown during inhalation by the patient.

FIG. 8B shows the wearable article of FIG. 8A during exhalation by the patient.

FIG. 9A shows another example embodiment of the wearable article that can be used by the monitoring system of FIG. 1 to monitor the respiration of the patient, the wearable article is shown during inhalation by the patient.

FIG. 9B shows the wearable article of FIG. 9A during exhalation by the patient.

FIG. 10 shows a cross-sectional view of the wearable article shown in FIGS. 9A-9B.

FIG. 11A shows another example embodiment of the wearable article that can be used by the monitoring system of FIG. 1 to monitor the respiration of the patient, the wearable article is shown during inhalation by the patient.

FIG. 11B shows the wearable article of FIG. 11A during exhalation by the patient.

FIG. 12 shows another example embodiment of the wearable article that can be used by the monitoring system of FIG. 1 to monitor the respiration of the patient.

FIG. 13 graphically illustrates an example of a curve generated from data acquired from the wearable article of FIG. 12.

FIG. 14 schematically illustrates an example of a method of monitoring respiration performed using the example embodiment of the wearable article shown in FIG. 12.

FIG. 15 shows another example embodiment of the monitoring system of FIG. 1.

### DETAILED DESCRIPTION

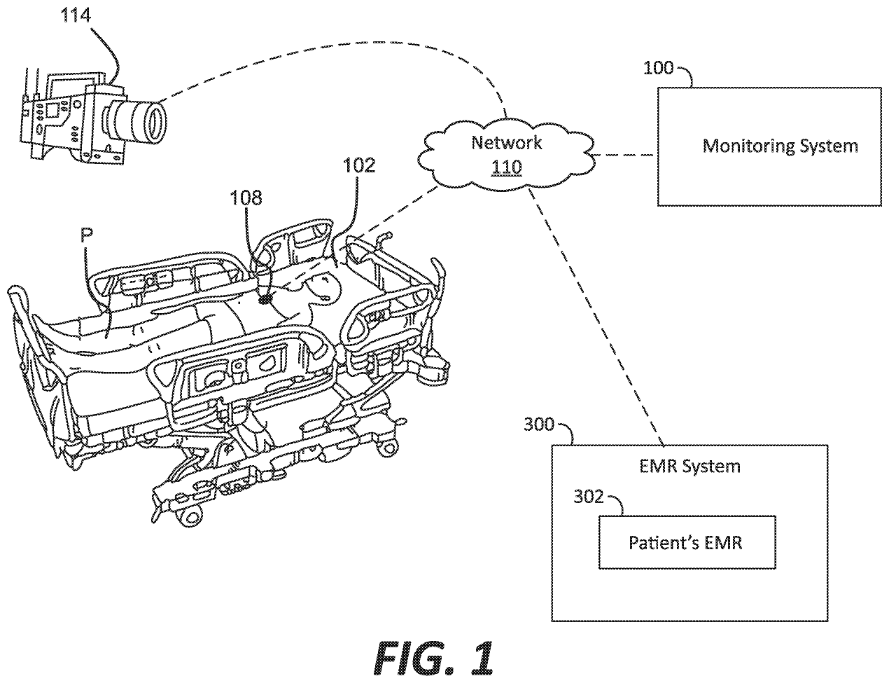

FIG. 1 illustrates an example of a monitoring system 100 that monitors one or more aspects of a patient P including at least the patient P's respiration. As will be described in more detail in the example embodiments described below, the monitoring system 100 utilizes a wearable article 108 worn by a patient P to monitor the patient P's respiration.

In some examples, the monitoring system 100 is a multi-parameter system that receives and analyzes data for monitoring multiple aspects of the patient P including the respiration of the patient P. In some examples, the monitoring system 100 is a spot monitor, such as the Connex® Spot Monitor, available from Hill-Rom®, Inc. of Batesville, Ind. In alternative examples, the monitoring system 100 is a single-parameter system that receives and analyzes data for monitoring only the patient P's respiration. In further examples, the monitoring system 100 is a server that is remotely located with respect to the patient P, and that processes and analyzes data from the wearable article 108 for monitoring the patient P's respiration.

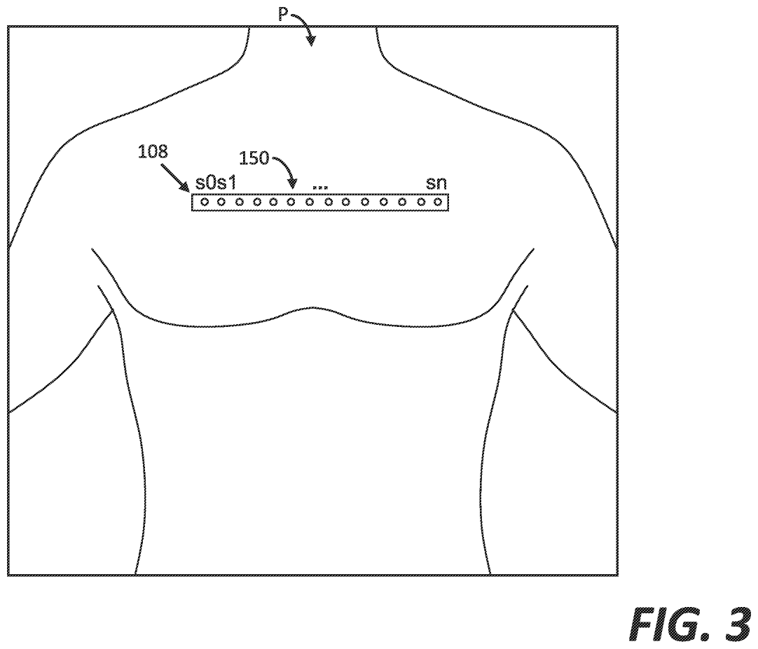

In one example embodiment, the monitoring system 100 is communicatively connected to the wearable article 108. In this example embodiment, the wearable article 108 includes a sensor array (see FIG. 3) that collects spatiotemporal data from the patient P that can be analyzed by the monitoring system 100 for monitoring the patient P's respiration. This example embodiment will be described in more detail with reference to FIGS. 3-7.

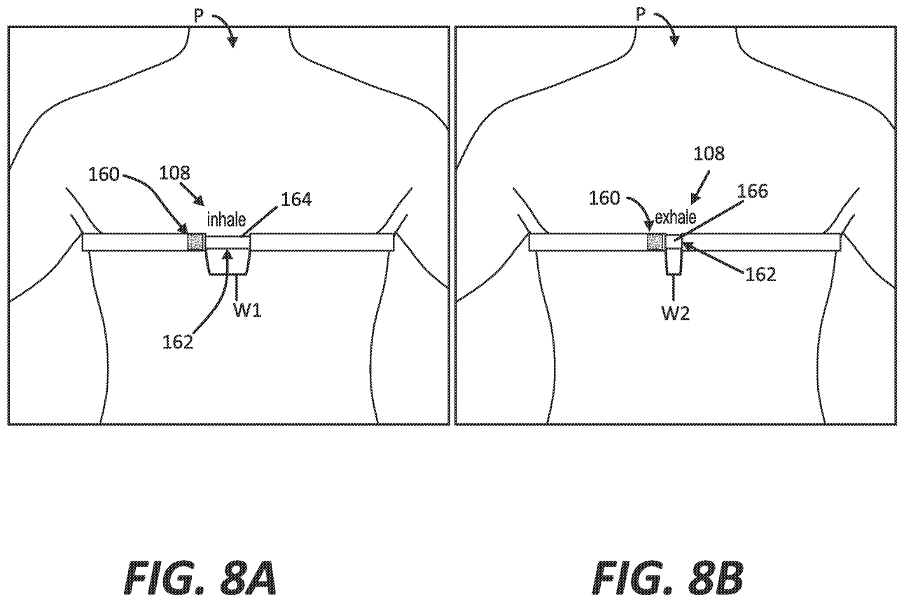

In another example embodiment, the monitoring system 100 is communicatively connected to a camera 114 that captures data such as video images of the wearable article 108 worn by the patient P. In this example embodiment, the wearable article 108 includes one or more markers that can be tracked by the monitoring system 100 for monitoring the patient P's respiration. This example embodiment will be described with reference to FIGS. 8-12.

In the example shown in FIG. 1, the patient P is shown resting on a patient support system 102. In this example, the patient support system 102 is illustrated as a hospital bed. In other examples, the patient support system 102 is another type of bed, lift, chair, wheelchair, stretcher, surgical table, and the like, which can support the patient P.

In the example shown in FIG. 1, the wearable article 108 and the camera 114 each communicate with the monitoring system 100 via a network 110. The network 110 can include any type of wired or wireless connections or any combinations thereof. Examples of wireless connections include Wi-Fi, Bluetooth, ultra-wideband (UWB), radio frequency identification (RFID), cellular network connections, and other wireless connections. In some examples, the network 110 is an Internet-of-things (IoT) network that connects and exchanges data between a plurality of devices and systems over the Internet or other communications networks.

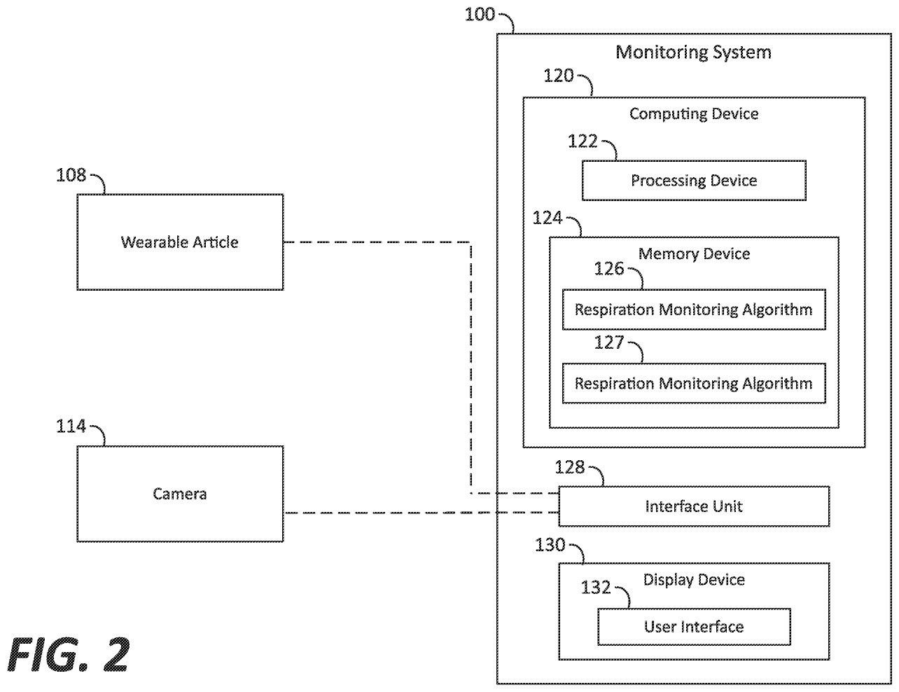

In alternative examples, the wearable article 108 and/or the camera 114 directly communicate with the monitoring system 100 without using the network 110. For example, the wearable article 108 can directly communicate with the monitoring system 100 via a wireless or wired connection, as shown in FIG. 2. Similarly, the camera 114 can directly communicate with the monitoring system 100 via a wireless or wired connection, as shown in FIG. 2.

The monitoring system 100 can communicate with an electronic medical record (EMR) (alternatively termed electronic health record (EHR)) system 140 via the network 110. The EMR system 140 operates to manage the patient P's medical history and information. The EMR system 140 can be operated by a healthcare service provider, such as a hospital or medical clinic. The monitoring system 100 sends respiration measurements derived from data acquired from at least one of the wearable article 108 and the camera 114 to the EMR system 140 via the network 110. The EMR system 140 stores the respiration measurements in an electronic medical record (EMR) (alternatively termed electronic health record (EHR)) 142 of the patient P.

FIG. 2 schematically illustrates another example of the monitoring system 100. In one example embodiment, the wearable article 108 communicatively connects to the monitoring system 100 for transferring data that can be used to monitor the respiration of the patient P. In another example embodiment, the camera 114 communicatively connects to the monitoring system 100 for transferring data that can be used to monitor the respiration of the patient P.

The monitoring system 100 includes a computing device 120 having at least one processing device 122 and a memory device 124. The at least one processing device 122 can include one or more central processing units (CPUs), digital signal processors, field-programmable gate arrays, and/or other programmable electronic circuits.

The memory device 124 operates to store data and instructions for execution by the at least one processing device 122. The memory device 124 stores at least a first respiration monitoring algorithm 126 and a second respiration monitoring algorithm 127 that can be performed by the at least one processing device 122 to monitor the patient P's respiration. The memory device 124 includes computer-readable media, which may include any media that can be accessed by the monitoring system 100. By way of example, computer-readable media include computer readable storage media and computer readable communication media.

Computer readable storage media includes volatile and nonvolatile, removable, and non-removable media implemented in any device configured to store information such as computer readable instructions, data structures, program modules, or other data. Computer readable storage media can include, but is not limited to, random access memory, read only memory, electrically erasable programmable read only memory, flash memory, and other memory technology, including any medium that can be used to store information that can be accessed by the monitoring system 100. The computer readable storage media is non-transitory.

Computer readable communication media embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are within the scope of computer readable media.

The monitoring system 100 includes an interface unit 128 that can operate to directly communicate with various sensors and/or devices including at least the wearable article 108 and/or the camera 114. In further examples, the interface unit 128 operates to communicate with the network 110. The interface unit 128 can include both wired interfaces and wireless interfaces. The wearable article 108 and/or the camera 114 can wirelessly connect to the interface unit 128 through Wi-Fi, ultra-wideband (UWB), Bluetooth, and similar types of wireless connections. Alternatively, the wearable article 108 and/or the camera 114 can connect to the monitoring system 100 using wired connections that plug into the interface unit 128.

In some examples, the monitoring system 100 includes a display device 130, which operates to display a user interface 132. In some examples, the display device 130 is a touchscreen such that the user interface 132 operates to display outputs and receive inputs from a clinician. In such examples, the display device 130 operates as both a display device and a user input device. The monitoring system 100 can also support physical buttons on a housing to receive inputs from the clinician to control operation of the monitoring system and enter data.

FIG. 3 shows an example embodiment of the wearable article 108 used by the monitoring system 100 to monitor the respiration of the patient P. In this example, the wearable article 108 includes a sensor array 150 that enables spatiotemporal modeling for high accuracy monitoring of the patient P's respiration activity. The sensor array 150 includes a plurality of sensors S₀, S₁ . . . . Sₙ arranged in a spatial configuration. Illustrative examples of the plurality of sensors S₀, S₁ . . . Sₙ include capacitive sensors, strain gauge sensors, radio mmWave sensors, impedance sensors, and the like. In further examples, the plurality of sensors S₀, S₁ . . . Sₙ can include visual markers, which are described in more detail in the embodiments discussed below. The sensor array 150 detects spatiotemporal data points of a single channel, or multiple channels such as when multiple types of sensors are included in the plurality of sensors S₀, S₁ . . . Sₙ.

The spatial configuration of the sensor array 150 may vary based on the types of sensors included in the plurality of sensors S₀, S₁ . . . Sₙ. In the example shown in FIG. 3, the spatial configuration is linearly arranged across the chest of the patient P. In further examples, the sensor array 150 can include one or more rows of sensors and one or more columns of sensors. Also, the relative positioning of the sensors with respect to one another in the sensor array 150 may vary. Also, the sensors in the sensor array 150 can be arranged in different geometric shapes such as rectangular, elliptical, circular, and the like. Thus, the spatial configuration of the sensor array 150 shown in FIG. 3 is provided as an illustrative example.

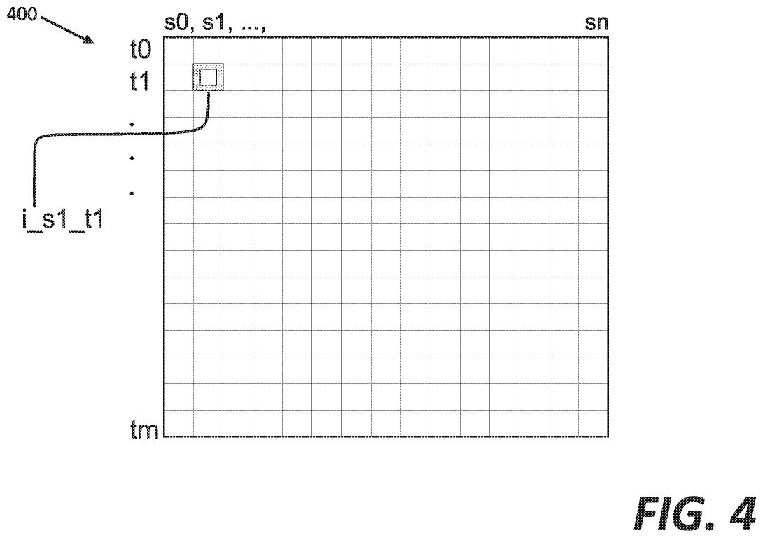

FIG. 4 illustrates an example of a chart 400 illustrating spatiotemporal data detected by the sensor array 150 of the wearable article 108. The spatiotemporal data include data points that are collected across space (x-axis) and time (y-axis). Each data point in the in the spatiotemporal data includes at least one spatial component (Sₙ) and at least one temporal component (tₘ). For example, the spatiotemporal data input i_S1_t1 represents an input from sensor S1 at time t1. The spatiotemporal data can include capacitive, impedance, pressure, mm Wave measurements, and the like depending on the types of sensors included in the plurality of sensors S₀, S₁ . . . Sₙ. In FIG. 4, the spatiotemporal data is a N×M matrix.

When the monitoring system 100 receives the spatiotemporal data from the wearable article 108, the monitoring system 100 can input the spatiotemporal data into the first respiration monitoring algorithm 126 stored in the memory device 124 for generating one or more outputs for high accuracy monitoring of the patient P's respiration activity.

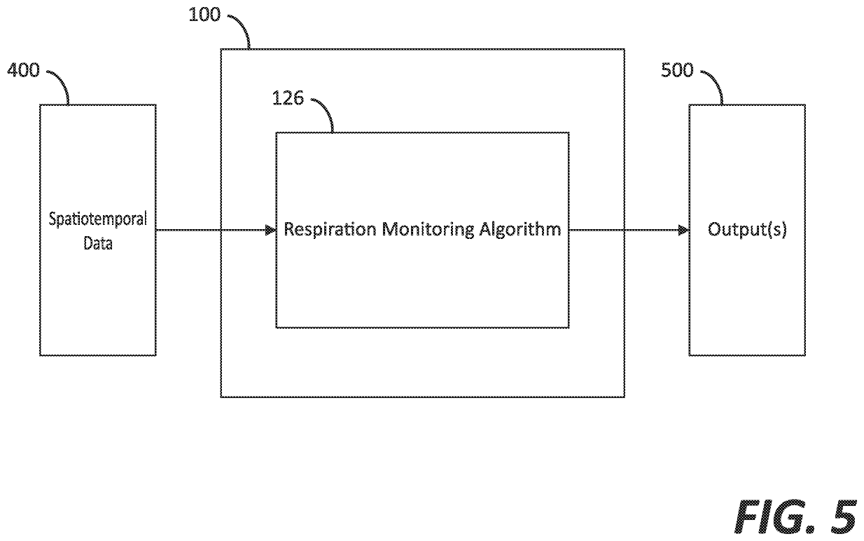

FIG. 5 illustrates an example of the first respiration monitoring algorithm 126 stored in the monitoring system 100. The first respiration monitoring algorithm 126 is a machine learning model that determines one or more outputs 500 for characterizing the respiration of the patient P. In some examples, the machine learning model is a deep learning artificial neural network that is supervised, semi-supervised, or unsupervised. In some examples, the machine learning model is a convolutional neural network (CNN), or similar type of neural network.

The monitoring system 100 enters the spatiotemporal data into the first respiration monitoring algorithm 126 for determining one or more outputs 500 such as a bounding box regression that can be used to measure the respiration rate of the patient P. In examples where the first respiration monitoring algorithm 126 includes a CNN or similar type of neural network model, the model learns the bounding boxes of breath over two dimensional inputs (e.g., the spatiotemporal data) to determine timing and relative volume changes associated with each breath. In this manner, the model can also learn different types of breathing such as normal breathing and abnormal breathing, which is a deviation from normal breathing.

In the examples shown in FIGS. 4 and 5, the spatiotemporal data is shown as including a single channel for the sensor array 150. In further examples, the spatiotemporal data can include multiple channels. For example, the spatiotemporal data can include multiple channels when a plurality of the sensor arrays 150 are positioned on the patient P. As an illustrative example, a first sensor array can be positioned on the front chest of the patient P, a second sensor array can be positioned on the back of the patient P (e.g., opposite the first sensor array), and a third sensor array can be positioned around the abdomen of the patient P. In such example, the spatiotemporal data includes three channels, one for each sensor array.

In further examples, the spatiotemporal data includes multiple channels when the wearable article 108 includes multiple sensor types. For example, the wearable article 108 can include multiple sensor arrays each having a unique sensor modality such as motion, impedance, capacitive, strain gauge, mmWave, and the like. In such examples, the spatiotemporal data includes a channel for each sensor modality of the multiple sensor arrays.

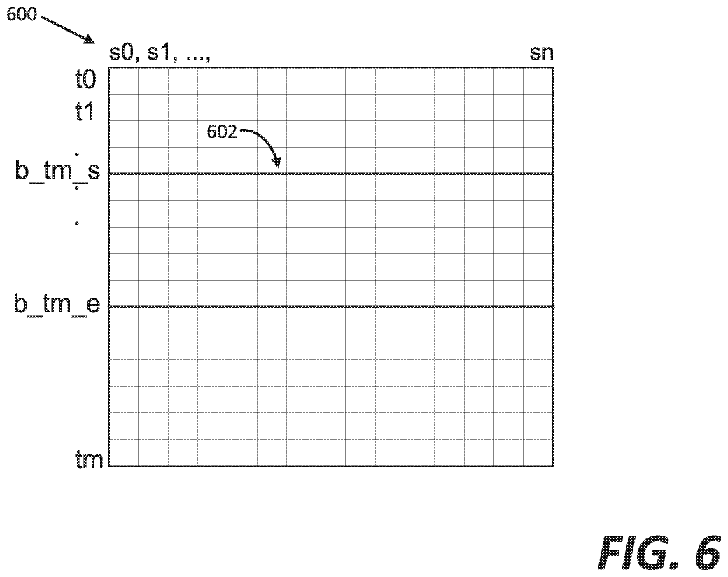

FIG. 6 graphically illustrates an example of a chart 600 having a bounding box 602 generated as an output 500 from the first respiration monitoring algorithm 126 shown in FIG. 5. The bounding box 602 detects a breath of the patient P at time tm (b_tm). The bounding box 602 includes at least two parameters along the time dimension (y-axis). For example, the bounding box 602 can define a start time (b_tm_s) and an end time (b_tm_e) for a breath of the patient P.

Multiple bounding boxes can be generated as outputs 500 of the first respiration monitoring algorithm 126 to detect breaths of the patient P over a period of time. In some examples, a weighted average length (bl_tm) of the breaths is determined over the period of time, and a breath per minute (BPM) is calculated as 60/bl_tm. Also, the bounding boxes can be used to measure the quality of breathing (e.g., shallow breathing), lung volume during inhalation, and other parameters related to respiration. In this manner, the monitoring system 100 can monitor the respiration activity of the patient P with a high level of accuracy.

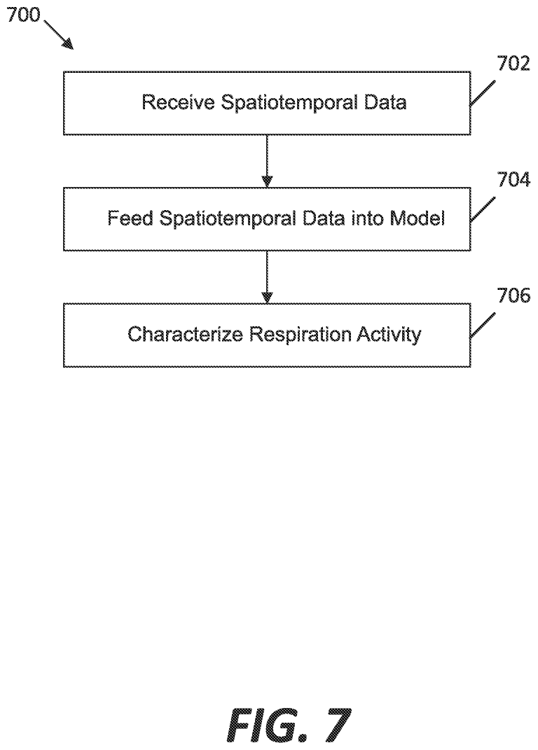

FIG. 7 schematically illustrates an example of a method 700 of monitoring respiration that can be performed by the monitoring system 100. In certain examples, the method 700 is performed by the monitoring system 100 based on the data received from the sensor array 150 of the wearable article 108, as shown in the example embodiment of FIG. 3.

The method 700 includes an operation 702 of receiving spatiotemporal data from the sensor array 150 of the wearable article 108. As discussed above, the spatiotemporal data includes data points each having at least one spatial parameter (Sₙ) and at least one temporal parameter (tₘ). The spatiotemporal data is a two-dimensional matrix of data points. In some examples, the spatiotemporal data received in operation 702 is from a single channel. In other examples, the spatiotemporal data received in operation 702 is from multiple channels.

Next, the method 700 includes an operation 704 of feeding the spatiotemporal data into a machine learning model. In some examples, the machine learning model is a convolutional neural network (CNN), or similar type of neural network. In some examples, the machine learning model utilizes bounding boxes to identify a start time and an end time for each breath.

Next, the method 700 includes an operation 706 of characterizing the respiration activity based on one or more outputs generated from the machine learning model. For example, operation 706 can include determining at least one of a weighted average length of breath over a period of time, breaths per minute (BPM) for determining a respiration rate of the patient, lung volume during inhalation, and other parameters related to respiration activity.

In the following example embodiments, the wearable article 108 assists video-based respiration monitoring performed using data captured by the camera 114. In some examples, the wearable article 108 and the camera 114 are part of a video monitoring system such as a virtual nursing system. In some examples, aspects of the video monitoring system are performed by the monitoring system 100 that receives the data captured by the camera 114. As will be described in more detail, the wearable article 108 includes one or more markers that are identified and monitored by the monitoring system 100 for monitoring the respiration of the patient P.

The one or more markers included on the wearable article 108 do not require power, are non-invasive, and do not transfer data. Instead, the one or more markers are detectable in the data captured by the camera 114. For example, the second respiration monitoring algorithm 127 stored in the memory device 124 of the monitoring system 100 can detect and monitor the one or more markers on the wearable article 108 for monitoring the respiration of the patient P.

FIGS. 8A and 8B show an example embodiment of the wearable article 108. In FIG. 8A, the wearable article 108 is shown when the patient P inhales. In FIG. 8B, the wearable article 108 is shown when the patient P exhales. In this example embodiment, the wearable article 108 is worn around the chest of the patient P, and is configured to deform (e.g., stretch) during respiration by the patient P. In some examples, the wearable article 108 is a band made of an elastic material such as nylon fabric or similar types of fabric material. The wearable article 108 stretches when the patient P inhales (e.g., due to expansion of the chest cavity), and the wearable article 108 contracts when the patient P exhales (e.g., due to contraction of the chest cavity). The wearable article 108 includes a first marker 160 and a second marker 162.

The first marker 160 is a machine-readable label that includes data that identifies the patient P. For example, the machine-readable label can include data that identifies a name, a number (e.g., medical record number), or a location of the patient P. In some examples, the first marker 160 includes a bar code, a quick response (QR) code, or similar type of label.

The second marker 162 changes properties when the wearable article 108 deforms during respiration by the patient P. For example, the second marker 162 can include a mechanochromic material that changes reflection and absorption of light due to mechanical action of the wearable article 108, such as when the wearable article 108 stretches during inhalation by the patient P and when the wearable article 108 contracts during exhalation. In some examples, the mechanochromic material is a polymer-based material.

The mechanochromic material causes the second marker 162 to change properties during respiration by the patient P. For example, the mechanochromic material can include one or more dyes that cause the second marker 162 to change color in response to the mechanical action of the wearable article 108 during respiration by the patient P. In some examples, the one or more dyes include luminescent dyes that are visible in the dark when the patient P is sleeping. In some further examples, the second marker 162 includes a mechanoluminescence material that causes light emission resulting from mechanical action on the wearable article 108.

FIG. 8A shows the second marker 162 having a first width W1 which is caused by the expansion of the patient P's chest cavity during inhalation. In this example, the one or more dyes in the mechanochromic material cause the second marker 162 to have a first color 164 (e.g., blue) when the patient P inhales. The first color 164 is recognized by the monitoring system 100 for identifying when the chest cavity of the patient P expands during inhalation.

FIG. 8B shows the second marker 162 having a second width W2 which is caused by the contraction of the patient P's chest cavity when the patient P exhales. The second width W2 is less than the first width W1 (FIG. 8A). The one or more dyes in the mechanochromic material cause the second marker 162 to have a second color 166 (e.g., yellow) when the patient P exhales. The second color 166 is different from the first color 164. The second color 166 is recognized by the monitoring system 100 for identifying when the chest cavity of the patient P contracts during exhalation for monitoring the patient P's respiration.

The monitoring system 100 can track the transitions between the first and second colors 164, 166 of the second marker 162 to monitor the patient P's respiration. For example, the camera 114 can continuously capture data such as video images of the wearable article 108 during respiration by the patient P. The second respiration monitoring algorithm 127 uses the data captured by the camera 114 to track the transitions between the first and second colors 164, 166 of the second marker 162 to monitor the respiration of the patient P.

The second respiration monitoring algorithm 127 monitors the color changes of the second marker 162 to determine respiration rate (e.g., breaths per minute), weighted average length of breath, lung volume during inhalation, and/or additional parameters related to respiration. For example, the second respiration monitoring algorithm 127 can monitor a degree of color change between the first and second colors 164, 166 to determine lung volume during inhalation by the patient P, where stronger color contrasts between the first and second colors 164, 166 indicate larger lung volume, and where weaker color contrasts between the first and second colors 164, 166 indicate smaller lung volume. The contrast between the first and second colors 164, 166 can also be used to monitor abnormal breathing such as shallow breathing.

In this embodiment, the wearable article 108 can improve the functioning of the monitoring system 100 because monitoring the transitions between the first and second colors 164, 166 requires simple processing for determining the respiration rate of the patient P. Thus, the wearable article 108 can free up processing capacity in the monitoring system 100 for performing other tasks in addition to monitoring the respiration of the patient P.

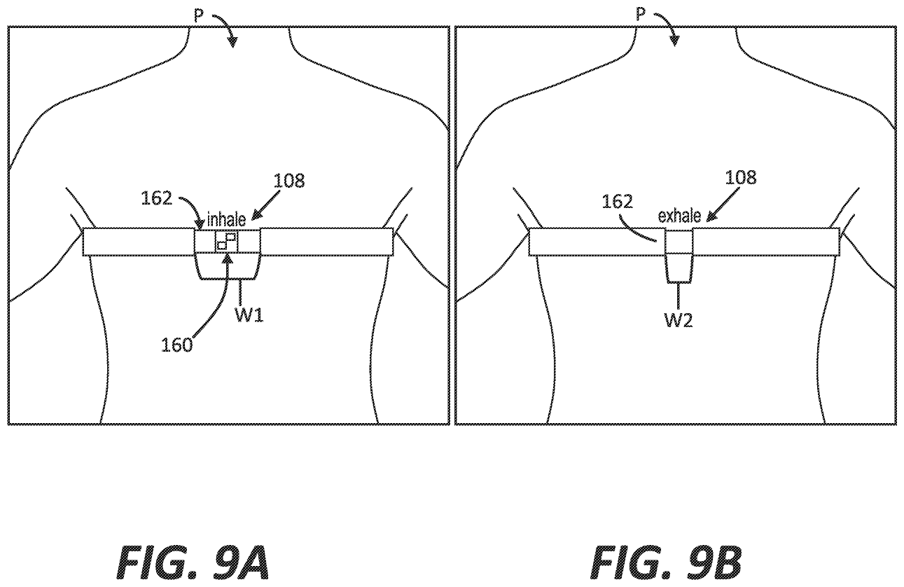

FIGS. 9A and 9B show another example embodiment of the wearable article 108. In FIG. 9A, the wearable article 108 is shown when the patient P inhales. In FIG. 9B, the wearable article 108 is shown when the patient P exhales. Like in the example embodiment described above, the wearable article 108 shown in FIGS. 9A and 9B can be used to assist video-based respiration monitoring that is performed using data captured by the camera 114.

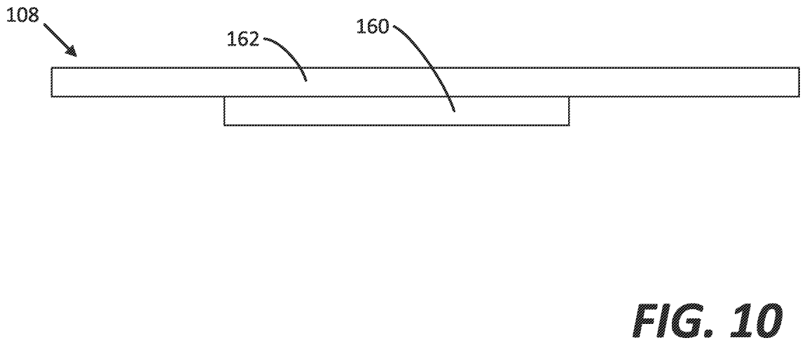

FIG. 10 shows a cross-sectional view of the wearable article 108 shown in FIGS. 9A and 9B. In this example embodiment, the second marker 162 is an elastomer layer covering the first marker 160, which is a patterned reflector layer. The second marker 162 includes a layer of mechanochromic material that covers the first marker 160. The second marker 162 changes properties when the wearable article 108 deforms during respiration by the patient P.

In this example, the mechanochromic material of the second marker 162 changes opacity due to mechanical action of the wearable article 108, such as when the wearable article 108 stretches during inhalation by the patient P and when the wearable article 108 contracts during exhalation. For example, when the wearable article 108 is stretched during inhalation, high density cracks form in the second marker 162 allowing visibility of the first marker 160. When released during exhalation, the first marker 160 is hidden by the second marker 162.

FIG. 9A shows the second marker 162 having a first width W1 which is caused by stretching of the wearable article 108 due to the expansion of the patient P's chest cavity when the patient P inhales. The mechanochromic polymer causes the second marker 162 to be transparent and thereby allows visibility of the first marker 160 which is positioned under the second marker 162. Thus, the first marker 160 is readable by the monitoring system 100 each time the patient P inhales, which causes the wearable article 108 to stretch.

In some examples, the first marker 160 is a machine-readable label such as a bar code, a quick response (QR) code, or similar type of machine-readable label that includes data that identifies the patient P. In some examples, the first marker 160 includes a patterned reflector layer that is luminescent such that it is visible in the dark when the patient P is sleeping.

FIG. 9B shows the second marker 162 having a second width W2 which is caused by the contraction of the patient P's chest cavity when the patient P exhales. The second width W2 is less than the first width W1. The mechanochromic material causes the second marker 162 to be opaque such that it conceals the first marker 160. When concealed each time the patient P exhales, the first marker 160 is not readable by the monitoring system 100.

In this manner, the monitoring system 100 can track the appearance and the disappearance of the first marker 160 to monitor the patient P's respiration. For example, the second respiration monitoring algorithm 127 stored in the memory device 124 of the monitoring system 100 can track the appearance and the disappearance of the first marker 160 to determine respiration rate (e.g., breaths per minute), weighted average length of breath, lung volume during inhalation, and/or additional parameters related to respiration.

In this embodiment, the wearable article 108 can improve the functioning of the monitoring system 100 because tracking the appearance and the disappearance of the first marker 160 requires simple processing for determining the respiration rate of the patient P. Thus, the wearable article 108 can free up processing capacity in the monitoring system 100 for performing other tasks in addition to monitoring the respiration of the patient P.

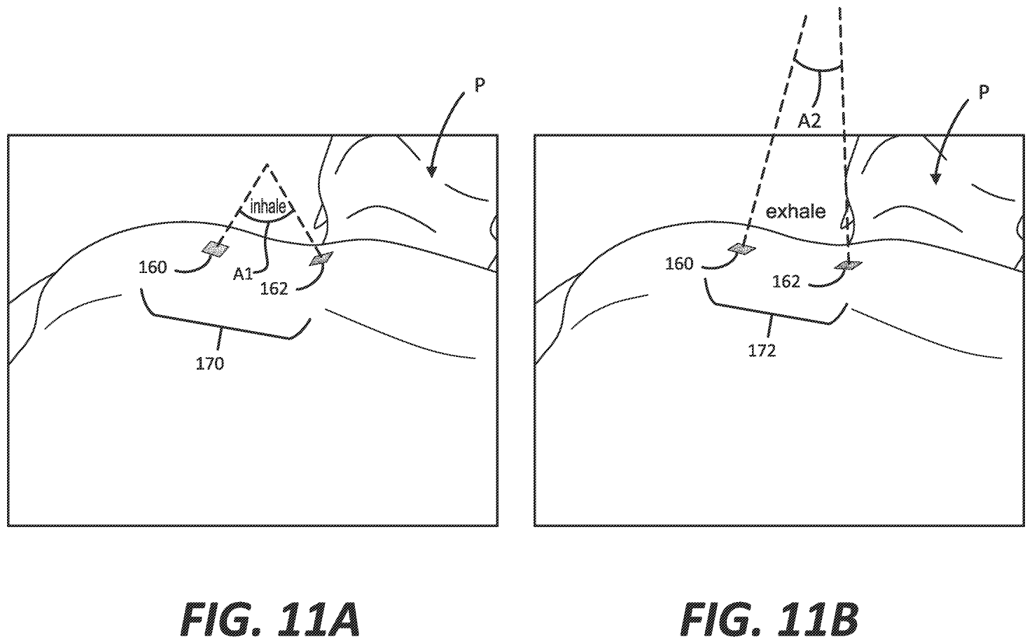

FIGS. 11A and 11B show another example embodiment of the wearable article 108. In FIG. 11A, the wearable article 108 is shown when the patient P inhales. In FIG. 11B, the wearable article 108 is shown when the patient P exhales. Like in the embodiments described above, the wearable article 108 shown in FIGS. 11A and 11B can be used to assist video-based respiration monitoring that is performed using data captured by the camera 114.

In this example, the first and second markers 160, 162 are a reference point for identifying the patient P's chest. Also, the first and second markers 160, 162 are orientated such that changes in the relative angle between the first and second markers 160, 162 can be used to monitor movements of the patient P's chest during respiration.

In this example embodiment, the wearable article 108 includes an article of clothing worn by the patient P such as a garment, gown, shirt, and the like, or an article that covers the patient P's body such as a blanket. The first and second markers 160, 162 attach to the wearable article using a non-permanent, removable adhesive such as double-sided removable fabric tape, removable glue, hook and loop fastener, and similar adhesives.

In some examples, the first and second markers 160, 162 include a reflective material that is optimized for excitation and detection in the near-infrared spectrum. In some further examples, the first and second markers 160, 162 include a luminescent material such that the first and second markers 160, 162 are visible in the dark such as when the patient P is sleeping.

As shown in the example provided in FIG. 11A, the first and second markers 160, 162 have a first orientation 170 when the patient P inhales. In the first orientation 170, the first and second markers 160, 162 are angled in convergent directions such that the first and second markers 160, 162 have a first relative angle A1 between them when the patient P inhales.

As shown in the example of FIG. 11B, the first and second markers 160, 162 have a second orientation 172 when the patient P exhales. In the second orientation 172, the first and second markers 160, 162 have a second relative angle A2 that is less than the first relative angle A1. Thus, the relative angle between the first and second markers 160, 162 changes as the patient P inhales and exhales during respiration. The transitions between the first and second relative angles A1, A2 can be tracked for monitoring the respiration of the patient P.

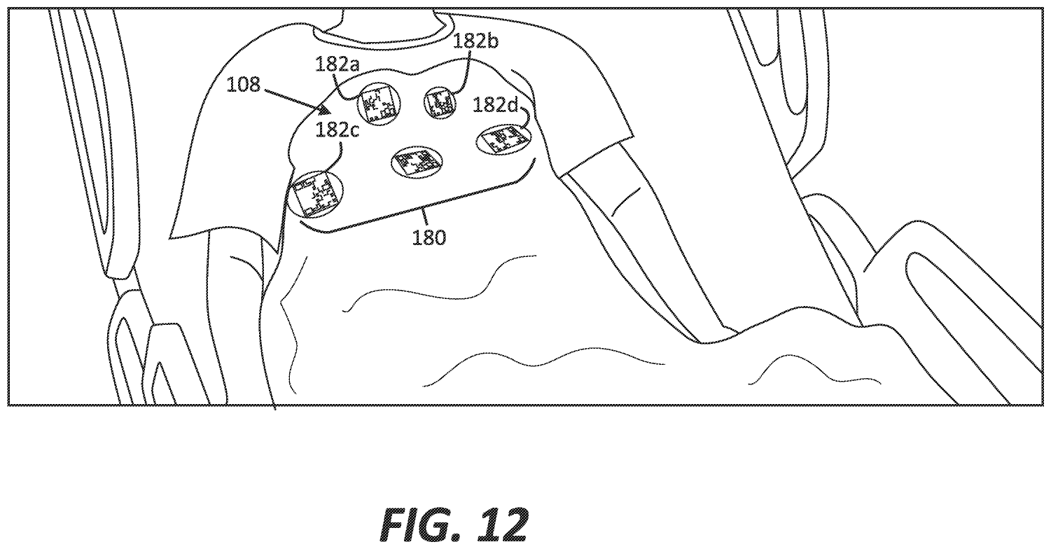

FIG. 12 shows another example embodiment of the wearable article 108 that can be used by the monitoring system 100 to monitor the respiration of the patient P. In this example embodiment, the wearable article 108 includes an article of clothing worn by the patient P such as a garment, gown, shirt, and the like, or an article that covers the patient P's body such as a blanket. The wearable article 108 includes a pattern 180 that includes one or more edges that can be tracked in the data captured by the camera 114 for monitoring the respiration of the patient P.

The pattern 180 may vary in size, shape, and configuration. In the example embodiment shown in FIG. 12, the pattern 180 includes four pixels 182a-182d that define the edges of the pattern. The number of pixels 182 included within the pattern 180 may vary.

The pattern 180 is used by the monitoring system 100 to identify one or more edges on the wearable article 108, and to thereafter track a frequency of motion of the one or more edges to determine the respiration rate of the patent P. In this embodiment, the pattern 180 can be printed, sprayed, sewn, or stitched onto the wearable article 108, or can be temporarily attached to the wearable article 108 using a non-permanent, removable adhesive such as double-sided removable fabric tape, removable glue, hook and loop fastener, and similar adhesives.

The pattern 180 can be made of a luminescent material such that the pattern 180 is visible in the dark when the patient P is sleeping. The pattern 180 can also be made of a material that is visible within a certain light frequency (e.g., infrared spectrum), and that is otherwise invisible to the human eye such that the pattern 180 is not noticeable to the patient P.

In some examples, the monitoring system 100 filters one or more frequencies detected from the wearable article 108 to identify a frequency most likely caused by the patient's respiration. For example, the monitoring system 100 can compare one or more frequencies detected from the wearable article 108 to a range that is typical for human respiration rate, and the monitoring system 100 ignores frequencies that are outside of the range.

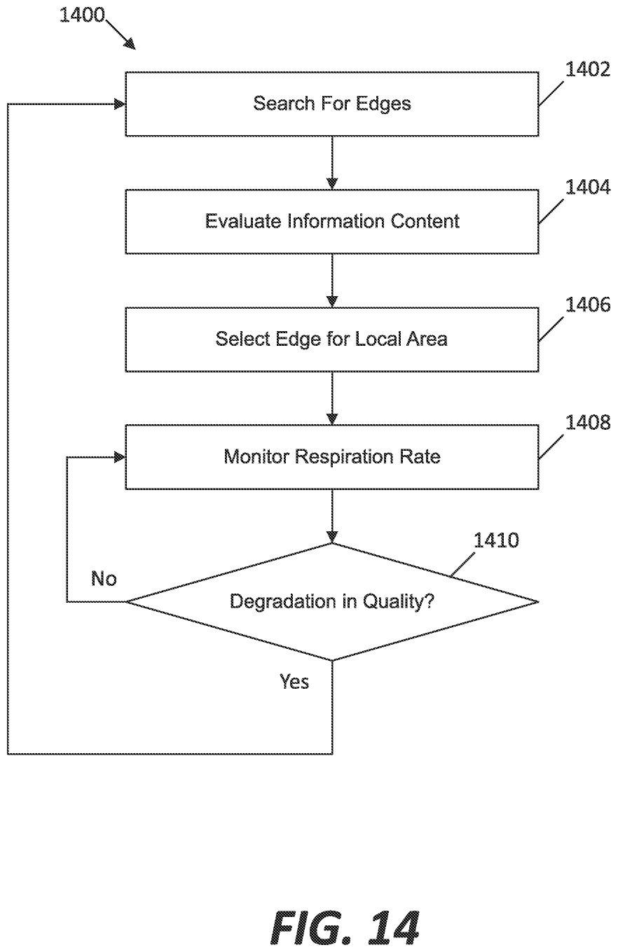

Also, the monitoring system 100 can distinguish the motion of the patient P's chest from movement of the patient P's body using the pattern 180 on the wearable article 108. For example, the pattern 180 can be used to identify a region of interest, and to track motion within the region of interest from a basepoint. This allows the monitoring system 100 to compensate the movement of the patient P's body when monitoring the patient P's respiration. Additional details on how to compensate the movement of the patient P's body will be described in more detail below with reference to the method 1400, which is schematically illustrated in FIG. 14.

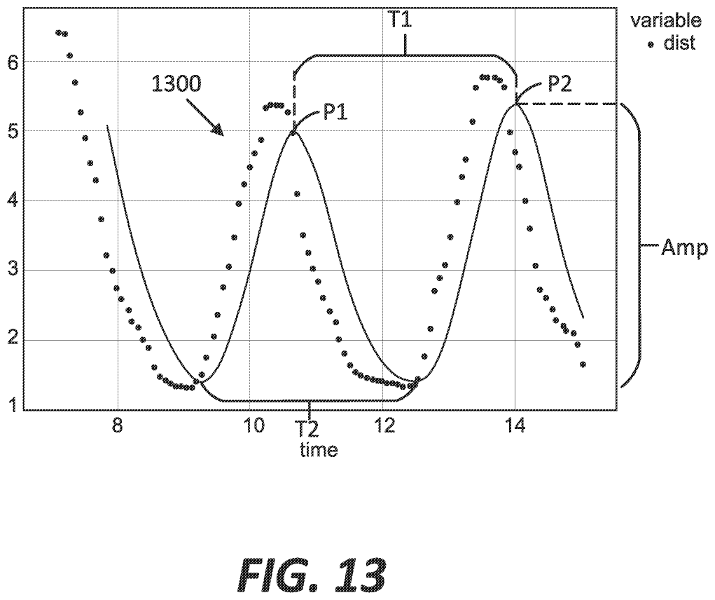

FIG. 13 graphically illustrates an example of a curve 1300 generated from data acquired from the camera 114 monitoring the wearable article 108 of FIG. 12 during respiration by the patient P. The curve 1300 can be generated by the second respiration monitoring algorithm 127 stored in the memory device 124 of the monitoring system 100.

As shown in FIG. 13, the second respiration monitoring algorithm 127 tracks movement of one or more edges on the wearable article 108 identified from the pattern 180 to generate the curve 1300. In some examples, the second respiration monitoring algorithm 127 is an optical flow algorithm that detects motion of the edges in the pattern 180 that are defined by the pixels 182 on the wearable article 108 to estimate the motion of the patient P's chest during respiration, which can be used to estimate the patient P's respiration rate.

In some examples, the second respiration monitoring algorithm 127 can be trained using the first respiration monitoring algorithm 126. For example, the data acquired from the sensor array 150 of the embodiment of the wearable article 108 described in FIGS. 3-7 has a higher quality and accuracy than the data acquired from first and second markers 160, 162 on the wearable article 108 shown in FIGS. 8-11, and the pattern 180 on the wearable article 108 shown in FIG. 12. To train the second respiration monitoring algorithm 127, the sensor array 150 and the first and second markers 160, 162 and/or the pattern 180 are worn by the patient P simultaneously. The outputs from the second respiration monitoring algorithm 127 based on tracking the first and second markers 160, 162 and/or the pattern 180 can be tracked to the outputs from the first respiration monitoring algorithm 126 to further refine and reduce noise from the second respiration monitoring algorithm 127, and thereby improve its accuracy.

The curve 1300 can be used to determine respiration rate (e.g., breaths per minute), weighted average length of breath, lung volume during inhalation, and/or additional parameters related to respiration. For example, the time (T1) between the peaks P1, P2 . . . . Pn of the curve can be used to determine respiration rate, the average time duration (T2) of each peak P1, P2 . . . . Pn can be used to determine an average length of breath, and the average amplitude (Amp) of the peaks P1, P2 . . . . Pn can be used to determine an average lung volume during inhalation.

Unlike other techniques for contactless respiration monitoring, the wearable article 108 of FIG. 12 does not require a clear image of the patient P's face, skin surface, or even their chest which can be covered such as in examples where the wearable article 108 is an article of clothing or a blanket that covers the patient P's chest. Instead, the monitoring system 100 can track one or more edges in the pattern 180 for determining the respiration rate of the patient P. This can improve respiration monitoring in scenarios where the patient P's chest is not visible.

FIG. 14 schematically illustrates an example of a method 1400 of monitoring respiration performed using the example embodiment of the wearable article 108 shown in FIG. 12. The method 1400 can adapt the monitoring system 100 to changing body position, angles, edge tracking error, and the like to mitigate the effects of the patient P's body movement when continuously or periodically measuring the respiration rate of the patient P.

The method 1400 includes an operation 1402 of searching for edges in the pattern 180 on the wearable article 108. In some examples, operation 1402 includes performing a global search for edges in the pattern 180. As shown in FIG. 12, the wearable article 108 can include a plurality of pixels 182 that define a plurality of edges in the pattern 180. The global search includes a search of the whole field of view of the camera 114 as its scope.

Next, the method 1400 includes an operation 1404 of evaluating information content of the edges identified in the search performed in operation 1402. The information content can include information such as a motion frequency of the edges.

Next, the method 1400 includes an operation 1406 of selecting at least one edge in the pattern 180 for identifying a local area of the pattern 180. The local area can include a plurality of edges that can be used for tracking respiration. The at least one edge can be selected in operation 1406 using a spectral density algorithm that includes checking the information content evaluated in operation 1406. For example, operation 1406 can include comparing a motion frequency of the edge to a motion frequency range typical for human respiration rate to determine whether the edge is appropriate for monitoring the respiration rate of the patient P.

In an alternative example, operations 1402-1406 can be replaced by an operation of performing a pose detection algorithm to locate the patient P's chest area, and using the results of the pose detection algorithm as the search scope for edge detection and selection.

Next, the method 1400 includes an operation 1408 of periodically or continuously monitoring the respiration rate of the patient P by evaluating the edges in the local area of the pattern 180 for information content in the respiration rate frequency range.

The method 1400 includes an operation 1410 of determining whether there is a degradation in quality of the content information acquired from the local area of the pattern 180. When the quality of the information content in the local area degrades, the method 1400 returns to operation 1402 to perform a local mask on the pattern 180. The local mask is a search limited to the local area for evaluating a new, optimal edge in the local area identified in operation 1406.

Otherwise, when the information content from the current edge does not degrade (i.e., “No” in operation 1410), the method 1400 can return to operation 1408 to periodically or continuously monitoring the respiration rate of the patient P by evaluating the at least one edge in the local area of the pattern 180 for information content in the respiration rate frequency range.

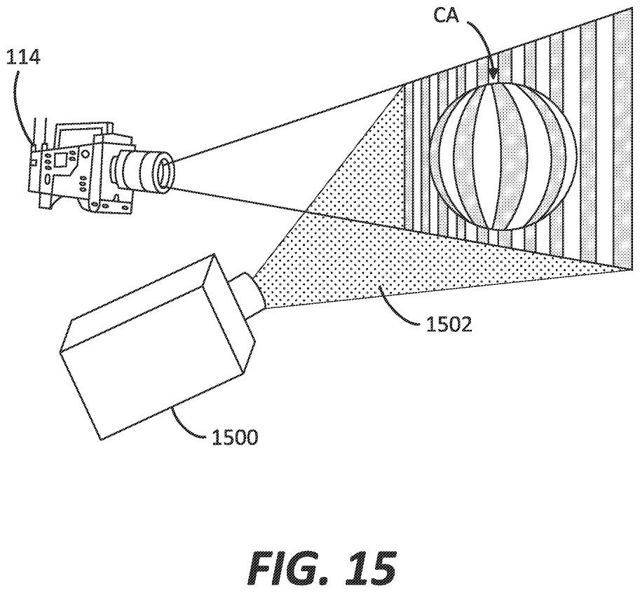

FIG. 15 shows another example embodiment of the monitoring system 100. In this example embodiment, a light projector 1500 can be a component of the monitoring system 100, or a component separate from the monitoring system 100 that is communicatively connected to the monitoring system 100 via the network 110 or a connection to the interface unit 128.

The monitoring system 100 can control the light projector 1500 to project a structured light 1502 onto the chest area CA of the patient P. The light projector 1500 projects the structured light 1502 to have a predetermined pattern on the chest area CA. For example, the structured light 1502 has a grid or horizontal bar pattern. The pattern of the structured light 1502 deforms when the structured light 1502 strikes the chest area CA, which allows the monitoring system 100 to calculate depth and surface information of the chest area CA.

In some examples, the light projector 1500 emits the structured light 1502 in a spectrum of light that is not visible by the patient P or other persons. In some examples, the light projector 1500 emits the structured light 1502 in the infrared spectrum.

The camera 114 captures data that tracks the deformation of the structured light 1502 on the chest area CA of the patient P while the patient P is breathing, and the camera 114 sends the data to the monitoring system 100 for monitoring the respiration of the patient P. For example, the depth and surface information of the chest area CA changes due to the expansion and contraction of the chest cavity of the patient P from breathing. This information can be processed by the second respiration monitoring algorithm 127 to monitor the respiration of the patient P. For example, the deformation of the pattern of the structured light 1502 can be processed to determine respiration rate (e.g., breaths per minute), weighted average length of breath, lung volume during inhalation, and/or additional parameters related to respiration.

The various embodiments described above are provided by way of illustration only and should not be construed to be limiting in any way. Various modifications can be made to the embodiments described above without departing from the true spirit and scope of the disclosure.

What is claimed is:

1. A system for monitoring respiration, the system comprising:
at least one processing device; and
at least one computer readable data storage device storing software instructions that, when executed by the at least one processing device, cause the at least one processing device to:
receive spatiotemporal data from a sensor array worn by a patient, the spatiotemporal data including spatial and temporal components;
feed the spatiotemporal data into a machine learning model; and
characterize the respiration based on an output of the machine learning model.

2. The system of claim 1, wherein characterizing the respiration includes determining at least one of a respiration rate, a weighted average length of breath, and a lung volume during inhalation.

3. The system of claim 1, wherein the machine learning model is a convolutional neural network.

4. The system of claim 1, further comprising:
a wearable article having the sensor array.

5. The system of claim 4, wherein the wearable article includes a plurality of sensor arrays, and the spatiotemporal data includes a plurality of channels associated with locations where the plurality of sensor arrays are positioned on the patient.

6. The system of claim 4, wherein the wearable article includes a plurality of sensor arrays, and the spatiotemporal data includes a plurality of channels associated with sensor modalities of the plurality of sensor arrays.

7. The system of claim 4, wherein the sensor array includes one or more rows of sensors, and further includes one or more columns of sensors.

8. A system for monitoring respiration, the system comprising:
at least one processing device; and
at least one computer readable data storage device storing software instructions that, when executed by the at least one processing device, cause the at least one processing device to:
identify one or more edges on a wearable article; and
track a frequency of motion of the one or more edges on the wearable device to determine a respiration rate.

9. The system of claim 8, further comprising:
the wearable article; and
wherein the one or more edges are included in a pattern that is printed, sprayed, sewn, stitched, or temporarily attached to the wearable article.

10. The system of claim 9, wherein the pattern includes a reflective material for excitation and detection in the near-infrared spectrum.

11. The system of claim 9, wherein the pattern includes a luminescent material.

12. The system of claim 8, further comprising:
the wearable article, wherein the wearable article includes first and second markers; and
wherein the instructions, when executed by the at least one processing device, further cause the at least one processing device to:
track changes in the first and second markers to characterize the respiration.

13. The system of claim 12, wherein the first marker is a machine-readable label, and wherein the second marker changes properties when the wearable article deforms during respiration.

14. The system of claim 13, wherein the second marker changes at least one of color and opacity when the wearable article deforms during respiration.

15. The system of claim 12, wherein the instructions, when executed by the at least one processing device, further cause the at least one processing device to:
track changes in a relative angle between the first and second markers to determine a respiration rate.

16. The system of claim 8, further comprising:
a light projector that projects a structured light onto a chest area; and
a camera that captures data tracking deformation of the structured light on the chest area due to expansion and contraction of a chest cavity from breathing; and
wherein the instructions, when executed by the at least one processing device, further cause the at least one processing device to:
process the data tracking deformation of the structured light on the chest area to determine a respiration rate of the patient.

17. A method of monitoring respiration, the method comprising:
searching for edges in a pattern on an article worn over a chest area;
evaluating information content of the edges;
selecting an edge in the pattern for identifying a local area of the pattern; and
monitoring the respiration by tracking a motion frequency of the local area of the pattern.

18. The method of claim 17, further comprising:
comparing a motion frequency of the edge in the pattern to a frequency range typical for human respiration to determine whether the edge is appropriate for monitoring the respiration.

19. The method of claim 17, wherein the local area includes a plurality of edges in the pattern.

20. The method of claim 17, further comprising:
when a quality of the information content from the local area degrades, performing a local mask on the pattern to identify a new local area for monitoring the respiration.
